(19) United States
(12) Patent Application Publication
(10) Pub. No.: US 2026/0199278 A1
(43) Pub. Date: Jul. 16, 2026

(54) NUTRITIONAL COMPOSITION FOR IMPROVING SOCIAL-EMOTIONAL DEVELOPMENT

(71) Applicant: SOCIÉTÉ DES PRODUITS NESTLÉ S.A., Vevey (CH)

(72) Inventors: Nora SCHNEIDER, Les Monts-de-Corsier (CH); Sean DEONI, Dighton, MA (US)

(21) Appl. No.: 19/129,815

(22) PCT Filed: Nov. 15, 2023
(86) PCT No.: PCT/EP2023/081928

§ 371 Date: May 14, 2025

(30) Foreign Application Priority Data
Nov. 15, 2022 (EP) 22207373.6

(51) Int. Cl. A61K 31/202; A23L 33/00; A61K 31/519; A61K 31/683; A61K 31/685; A61K 31/688; A61K 31/714; A61K 33/26; A61P 25/00

(52) U.S. Cl. CPC A61K 31/202; A23L 33/40; A61K 31/519; A61K 31/683; A61K 31/685; A61K 31/688; A61K 31/714; A61K 33/26; A61P 25/00

(57) ABSTRACT

The present invention relates to a combination of DHA, ARA, iron, folic acid, Vitamin B12 and phospholipids and to a nutritional composition comprising such a combination for use in improving social-emotional skills and/or in promoting mood, social and joy-related behaviours in an infant, a toddler or a young child. The present invention also relates to a synthetic nutritional composition comprising DHA, ARA, iron, folic acid, Vitamin B12 and phospholipids in specific amounts. The present invention further relates to uses and methods for improving social-emotional skills and/or for promoting mood, social and joy-related behaviours in an infant, a toddler or a young child.

### FIELD OF THE INVENTION

The present invention relates to a combination of DHA, ARA, iron, folic acid, Vitamin B12 and phospholipids and to a nutritional composition comprising such a combination for use in improving social-emotional skills and/or in promoting mood, social and joy-related behaviours in an infant, a toddler or a young child. The present invention also relates to a synthetic nutritional composition comprising DHA, ARA, iron, folic acid, Vitamin B12 and phospholipids in specific amounts. The present invention further relates to uses and methods for improving social-emotional skills and/or for promoting mood, social and joy-related behaviours in an infant, a toddler or a young child.

### BACKGROUND OF THE INVENTION

Breast feeding is considered as the ideal source of nutrition and is the preferred choice for feeding infants up to at least 6 months of age. Consequently, human milk (HM) has long been considered as the model for the design of infant formulas (IF). Even if many improvements in the nutrient composition of IF have been made during the last decades, there are still important differences in composition as well as in functional benefits conveyed by HM.

Identifying the potential relationship between human breast milk components and early brain development and associated functions has gained substantial interests in recent years.

Nutritional deficiencies, including iron, vitamin B12, and folate, have been associated with hypomyelination, altered myelin composition, or decreased myelin synthesis. Observational studies suggest early life differences between formula-fed and breast-fed infants in fatty acid related white matter composition as well as in myelination and cognitive abilities, possibly linked to long-chain poly-unsaturated fatty acids (LC-PUFA), sphingomyelin (SM), iron and folic acid levels in infant nutrition. (Schneider N, Hauser J, Oliveira M, et al. Sphingomyelin in Brain and Cognitive Development: Preliminary Data. eNeuro. 2019; 6(4):ENEURO.0421-18.2019. Published 2019 Aug. 6. doi:10.1523/ENEURO.0421-18.2019). The role of polar lipids in neurodevelopment, possibly via myelination has been reviewed in (Zheng L, Fleith M, Giuffrida F, O'Neill B V, Schneider N. Dietary Polar Lipids and Cognitive Development: A Narrative Review. Adv Nutr. 2019 Nov. 1; 10(6):1163-1176. doi: 10.1093/advances/nmz051. PMID: 31147721; PMCID: PMC6855982.). In our own past work (Hauser J, Sultan S, Rytz A, Steiner P, Schneider N. A blend containing docosahexaenoic acid, arachidonic acid, vitamin B12, vitamin B9, iron and sphingomyelin promotes myelination in an in vitro model. Nutr Neurosci. 2020 December; 23(12):931-945. doi: 10.1080/1028415X.2019.1580918. Epub 2019 Feb. 26. PMID: 30806182), we found that in vitro treatment with a blend of docosahexaenoic acid (DHA), arachidonic acid (ARA), vitamin B12, folic acid, iron and sphingomyelin in a primary cell culture model increased the number of oligodendrocyte precursor cells, their differentiation and maturation.

The relationship between human breast milk composition and its impact on the socio-emotional development in infants, toddlers and/or young children remains unclear, and there is no report in the prior art on the impact of a blend of nutrients comprising DHA, ARA, Vitamin B12m folic acid, iron and phospholipids on the socio-emotional development.

Social-emotional skills develop together from birth and during childhood and include behaviors that represent children's emotional growth and their ability to successfully interact with adults and peers. Socio-emotional milestones focus on children's developing abilities to understand, experience, express and manage emotions, regulate their emotions, actions and behavior in different social environment, and to develop meaningful relationships with others. As such, socio-emotional development encompasses a large range of skills and constructs, including but not limited to self-awareness, knowledge of others, autonomy, compliance, affect, sociability, play and interaction, empathy, self-esteem, self-regulation, friendship and relationship development.

Social skills include verbal and non-verbal communication and are vital in enabling an individual to have and maintain positive interactions with others. Children with a diagnosis of developmental disorders such as Autism Spectrum Disorders (ASD), Persuasive Developmental Disorder (Not otherwise Specified) and Asperger's, as well as diagnosis of mood or anxiety disorders such as Social Anxiety Disorder have difficulties with social skills or behaviors in social situations.

Forming social skills is an integral part of child's development. Social skills allow children interacting with the world around through understanding themselves and others, relationship development, verbal communication and body language. The right set of social skills will facilitate cooperation in social environment. Furthermore, well-developed social skills are foundational to the development of cognition, language, and adaptive life skills.

Another aspect of social-emotional development is the development of emotional skills and competencies, including mood. Emotional development refers to the ability to effectively regulate emotions to accomplish one's goal. Healthy emotional development therefore entails experiencing, managing, and expressing a full range of positive and negative emotions. It included behaviors such as the willingness to demonstrate own feelings as well as empathy for other, self-regulation or the ability to calm down or to adjust to the environment or stimulation.

Both social and emotional skills and behaviors mature alongside brain development, in particular brain regions and their connections to link to social and/or emotional information processing.

It is believed that lack of social and/or emotional skills can lead in poor outcomes in child development such as hindering child's ability to create relationship, affecting child's adjustment at school, influencing mood difficulties, leading to aggressive or disruptive behaviors, leading to loneliness and cause behavioral problems later in life. If preschoolers do not learn to identify and label their emotions for example, tantrums and inappropriate regulations of emotions may escalate later in life. In some instances, shy, inhibited children are at risk for later anxiety and depression, or behavior or conduct problems. Anxiety disorders are a cluster if mental disorders characterized by significant and uncontrollable feelings of anxiety and fear such that person's social, occupational, and personal function are significantly impaired. Many anxiety disorders have their onset in early childhood (Clark C, Rodgers B, Caldwell T, Stansfeld S. Childhood and adulthood psychological ill health as predictors of affective and anxiety disorders: the 1958 British Cohort. British Birth Cohort. Arch Gen Psychatry. 2007; 64:668-678; doi: 10.1001/archpsyc.64.6.668; Mian N, Godoy L, Briggs-Gowan M J, Carter A S. Patterns of anxiety symptoms in toddlers and preschool-age children: evidence of early differentiation. J. Anxiety Disord. 2012 January; 26(1): 102-110; doi: 10.1016/j.janxdis.2011.09.006.Epub 2011 Oct. 7). Examples of anxiety disorders include generalized anxiety disorder, social anxiety disorder (social phobia) and separation disorder. Generalized anxiety disorder, separation anxiety disorder, and social phobia are thought to be among the most common, earliest-onset childhood psychiatric disorders and depression in adulthood and for continuing comorbidity and impaired quality of life throughout the lifespan (Copeland W E, Shanahan L, Costello E J, Angold A. Childhood and adolescent psychiatric disorders as predictors of young adult disorders. Arch Gen Psychatry. 2009 July; 66(7): 764-772; doi: 10.1001/archgenpsychiatry.2009.85).

Mood disorders are a cluster of mental disorders that include for example bipolar and depressive disorders. Mood is defined as a pervasive and sustained feeling tone that is endured internally and which impacts nearly all aspects of a person's behavior in the external world. Mood disorders are described by marked disruptions in emotions (severe lows called depression or high called hypermania or mania). These include bipolar disorder, cyclothymia, hypomania, major depressive disorder, disruptive mood dysregulation, persistent depressive disorder, and premenstrual dysphoric disorder.

Conduct problems or disorders are diagnosed when children show an ongoing pattern of aggression toward others, and serious violation of rules and social norms at home, in school and with peers. Children with conduct problems are more likely to get injured and may have difficulties getting along with peers.

Current approach to avoid anxiety disorders and to promote development of social behavioral skills and social skills rely mainly on education of parents and caregivers, usually through early programs (Lau E, Rapee R, Coplan R. Combining child social skills training with a parent early intervention program for inhibited preschool children. Journal of Anxiety Disorders. 2017 October; 51:32-38; doi: 10.1016/j.janxdis.2017.08.007.Epub 2017 Sep. 1) to promote communication, promoting play, helping the child to understand emotions and develop empathy, telling stories and promoting group activities.

It has been reported a relationship between shy-inhibited behaviors (Prior M, Smart D, Sanson A, Oberklaid F. Does shy-inhibited temperament in childhood lead to anxiety problems in adolescence? J Am Acad Child Adolesc Psychatry. 2000 April; 39 (4): 461-8. doi: 10.1097/00004583-200004000-00015) or fearful behaviors (Liu R, Bell M. Fearful temperament and the risk for child and adolescent anxiety: the role of attention biases and effortful control. Clin Child Fam Psychol Rev. 2020 June; 23 (2): 205-228. doi: 10.1007/s10567-019-00306-z.) in childhood and the risk of developing anxiety problems in early adolescence.

Temperament broadly refers to consistent individual differences in behavior that are biologically based and are relatively independent of learning, system of values and attitudes. Some searchers point to association of temperament with formal dynamical features of behavior, such as energetic aspects, plasticity, sensitivity to specific reinforcers and emotionality. Temperament traits (such as Neuroticism, Sociability, impulsivity, etc) are distinct patterns in behavior throughout lifetime, but they are more noticeable and most studied in children. Temperament is defined through specific behavioral profiles, usually focusing on those that are both easily measurable and testable early in childhood. Commonly tested factors include traits related to energetic capacities (named as “Activity”, “Endurance”, “Extraversion”), traits related to emotionality (such as irritability, frequency of smiling), and approach or avoidance of unfamiliar events. Manifestation of temperament change over time as a child develops, with rapid development during infancy.

To the best of our knowledge, the present study is the first study reporting human results and investigating impact of a blend of certain myelinating nutrients present in human breast milk on the development of social-emotional skills, and/or on behaviors.

There is a need to deliver such benefit on social and emotional skills, and/or on behaviors in the subject in a manner that does not induce side effects and/or in a manner that is easy to deliver, and well accepted by the parents or health care practitioners. There is also a need to deliver such benefits in a manner that does keep the cost of such delivery reasonable and affordable by most.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

### SUMMARY OF THE INVENTION

The object of the present invention is achieved by the subject matter of the independent claims. The dependent claims further develop the idea of the present invention.

The inventors have surprisingly found that a combination or a nutritional composition comprising DHA, ARA, Vitamin B12m folic acid, iron and phospholipids administered to an infant, a toddler or a young child improves social-emotional skills and/or promotes mood and joy behaviors in the subjects.

Thus, in a first aspect the present invention provides a combination of DHA, ARA, iron, folic acid, Vitamin B12 and phospholipids for use in improving social-emotional skills and/or in promoting mood and joy behaviour in an infant, a toddler or a young child.

In a second aspect, the invention relates to a nutritional composition for use in improving social-emotional skills and/or in promoting mood and joy behaviour in an infant, a toddler or a young child, said composition comprising a combination of DHA, ARA, iron, folic acid, Vitamin B12 and phospholipids. The nutritional composition may be a synthetic nutritional composition.

A third aspect of the invention relates to the use a combination or of a nutrition al composition for improving social-emotional skills and/or for promoting mood and joy behaviour in an infant, a toddler or a young child

The present invention further provides a method for improving social-emotional skills and/or for promoting mood and joy behaviours in an infant, a toddler or a young child, the method comprising administering to said infant, toddler or young child a nutritional composition which comprises a combination of DHA, ARA, iron, folic acid, Vitamin B12 and phospholipids.

### BRIEF DESCRIPTION OF THE DRAWINGS

FIG. 1 is a Boxplot representation for sadness at 3, 6 and 12 months of age in investigational, control and breast-fed groups as described in the Example.

FIG. 2 is a Boxplot representation for high intensity pleasure at 3, 6 and 12 months of age in investigational, control and breast-fed groups as described in the Example.

FIG. 3 is a Boxplot representation for social fear at 18 and 24 months of age in investigational, control and breast-fed groups as described in the Example.

### DETAILED DESCRIPTION OF THE INVENTION

### Definitions

The term “subject” as used herein refers to a mammal and more particularly a cat, a dog or a human. In one embodiment, the subject is an infant, a toddler or a young child

The term “infant” means a child under the age of 12 months. The term infant includes both infants born at term or infant born preterm.

The terms “young child” or “toddler” mean a child aged between one and three years.

The term “child” means a child aged between one and 6 years, including toddlers and pre-school children.

The “IBQ-R (revised Infant Behavior Questionnaire) questionnaire” is a widely used parent-report measure for assessing dimensions of temperament along 14 scales, as reported in Table 1 below, in infants between 3 and 12 months of age (Garstein M A; Rothbart A K. Studying infant temperament via Revised Infant Behavior Questionnaire. Infant Behavior & Development 2003: 26: 64-86).

TABLE 1
Scale definitions: Infant Behavior Questionnaire -Revised

| IBQ-R scale | Scale description |
|---|---|
| Approach | Rapid approach, excitement, and |
| | positive anticipation of pleasurable |
| | activities. Amount of vocalization |
| | exhibited by the baby in daily activities. |
| Vocal | Pleasure and enjoyment related to high |
| Reactivity | stimulus intensity, rate, complexity, |
| | novelty, and incongruity. Smiling and |
| | laugher during general caretaking and |
| | play. |
| Activity | Gross motor activity, including |
| level | movement of arms and legs, squirming |
| | and locomotor activity. |
| Perceptual | Detection of slight, low intensity stimuli |
| Sensitivity | from the external environment. |
| Sadness | Lowered mood and activity related to |
| | personal suffering, physical state, object |
| | loss, or inability to perform desired |
| | action; general low mood. |
| Distress to | Fussing, crying or showing distress while |
| Limitations | (a) in a confining place or position; (b) in |
| | caretaking activities; (c) unable to |
| | perform a desired action. |
| Fear | Startle or distress to sudden changes in |
| | stimulation, novel physical objects or |
| | social stimuli; inhibited approach to |
| | novelty. |
| Falling | Rate of recovery from peak distress, |
| Reactivity/rate | excitement, or general arousal; ease of |
| of recovery | falling asleep. |
| from distress | |
| Low intensity | Amount of pleasure or enjoyment |
| pleasure | related to low stimulus intensity, rate |
| | complexity, novelty and incongruity. |
| High intensity | Pleasure and enjoyment related to high |
| pleasure | stimuli intensity, rate, complexity, |
| | novelty and incongruity |
| Cuddliness | Expression of enjoyment and molding of |
| | the body being held by a caregiver. |
| Duration of | Attention to and/or interaction with a |
| Orienting | single object for extended period of |
| | time. |
| Soothability | Reduction of fussing, crying, or distress, |
| | when soothing techniques are used by |
| | the caregiver. |
| Social Fear | Slow/inhibited approach and/or distress |
| | in social situations that involve |
| | novelty/uncertainty. |
| Attentional | The ability to transfer attentional focus |
| Shifting | from one activity/task to another. |

The “Toddler Behavior Assessment Questionnaire (TBAQ)” is used to measure behaviors in young children between 16 to 36 months (Goldsmith H H. Studying behaviors via construction of the toddler behavior assessment questionnaire. Child Development; 1996 February (67): 218-235. Doi: 10.2307/1131697). The TBAQ contains 120 items and 11 scales: activity level, perceptual sensitivity, inhibitory control, soothability, appropriate attentional allocation, sadness, object fearfulness, anger, social fearfulness, pleasure and interest. Items are rated on a 7-point scale ranging from 1=never to 4=about half the time to 7=always during the past month. A not applicable option is also provided for use when the respondent has not seen the child in the particular situation in the past month.

Within the context of the present invention, the terms “anxiety” and “fear” are used interchangeably. “Social fearfulness” refers to the developmental context of an inhibited approach and/or distress in social situations that involve novelty and/or uncertainty.

Within the context of the present invention, the expressions “social skill” or “social skills” encompass any competence or behavior observed in a social situation or environment and/or facilitating interaction and communication with others, both verbally and non-verbally, and development of social skills starts in the first few months of life. For example, infants begin to show signs of emotions, such as joy, anger, sadness, interest, fear, disgust and surprise as distinct facial expression. Infants begin also to laugh out and begin to socialize with people through babbling. Social skills continue developing in toddlers and children, including for example to begin imitating the environments around 15 months, clinging to caretakers in new situation (social fearfulness) at 18 months, parallel playing with other children at around 24 months, etc.

Within the context of the present invention, the expressions “social-emotional” or “social-emotional development” relates to social-emotional skills that develop from birth onwards allowing for increasing autonomy, self-regulation, and relationships.

Social-emotional development can be assessed and monitored behaviorally using clinical interviews, direct behavior observations, and rating systems as well as parent- and teacher-report questionnaires (Schneider N et al. Child Development 2022:93:359-371. Doi: 10.1111/cdev.13649).

Within the context of the present invention, the expressions “emotional skill” or “emotional skills” relate to the capacity to experience and express personal feelings, as well as recognize and interpret those of others. Emotional skills are linked for example to mood, pleasure, sadness. The development of emotional skills occurs in conjunction with neural, cognitive and behavioral development from birth throughout childhood, adolescence and adulthood.

Within the context of the present invention, the term “joy” refers to a feeling of great pleasure and happiness. Joy is a powerful antidote to stress which has negative effects during infant and child development. Ongoing stressful events trigger feelings of anxiety. Promoting joy may lessen anxious feelings.

Within the context of the present invention, the term “mood” refers to an emotional state of an individual at a particular time.

Within the context of the present invention, the expression “nutritional composition” refers to a composition which nourishes a subject. This nutritional composition is usually taken orally or intravenously. It may include a lipid or fat source, a carbohydrate source and/or a protein source. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults, porridges and/or infant cereals, baby food, pet food.

Within the context of the present invention, the expression “synthetic nutritional composition” refers to a mixture obtained by chemical and/or biological means that can be chemically identical to the mixture naturally occurring in mammalian milk (i.e., the synthetic nutritional composition is not breast milk).

Within the context of the present invention, the term “nutrient” or “nutrients” is intended to comprise both macronutrients (for example carbohydrates, proteins or fats) and micronutrients (for example minerals or vitamins) and components thereof (e.g. fatty acids, amino acids) for the human body.

Within the context of the present invention, the terms “ingredient” or “ingredients” indicate an edible substance or mixture of substances which comprise or is essentially consisting of a nutrient for the human body.

The terms “promote”, “promoting” and “promotion” can be used interchangeably. They should be understood as comprising support or help to the health of an individual, for example to support or help the development or growth of an individual. The individual may not suffer from a disease but may be susceptible to the development of unhealthy conditions, for example later in life.

Within the context of the present invention, the expression “reducing the risk of experiencing” or “reduce the risk of experiencing” should be understood as reducing the frequency or intensity of certain behavioral traits that are involved in temperament or sociability according to the IBQ-R and TABQ questionnaires. In one embodiment, the expression “reducing the risk of experiencing” comprises a decreased frequency and/or intensity of certain behavioral traits that are involved in temperament or sociability in the presence of a nutrient or number of nutrients.

Within the context of the present invention, the expressions “improve social-emotional skills” or “improve social skills” indicate an amelioration in social-emotional competences or behaviors in an infant, a toddler or a young child.

Within the context of the present invention, the expressions “in the prevention of”, “to prevent” or “for the prevention of” can be used interchangeably. They should be understood as comprising the decrease of the severity of a disease process. These expressions also encompass the relieve of the symptoms of induced by a disease, such as stress and/or decrease of complications caused by the disease.

Within the context of the present invention the expression “infant formula” refers to a foodstuff intended for particular nutritional use by infants during the first months of life satisfying by itself the nutritional requirements of this category of person (Article 20 of the European Commission Directive 91/321/EEC 2006/EC of 22 Dec. 2006 on infant formulae and on follow-on formulae). It also refers to a nutritional composition intended for infants and as defined in Codex Alimentarius (Codex STAN 72-1981) and Infant Specialties (incl. Food For Special Medical Purpose). The expression “infant formula” encompasses both “starter infant formula” and “follow-up formula” or “follow-on formula”.

The expressions “follow-up formula” or “follow-on formula” is given from the 6 months onwards and includes growing-up milk. It constitutes the liquid element in the progressively diversified diet of this category of person.

The expression “baby food” means a foodstuff intended for particular nutritional use by infants or young children during the first year of life.

The expression “infant cereal composition” means a foodstuff intended for particular nutritional use by the infants or young children during the two years of life.

The expression “growing-up milk (or GUM)” refers to a milk-based drink generally with added vitamins and minerals that is intended for young children or children.

The expression “fortifier” refers to liquid or solid nutritional composition suitable for fortifying or mixing with human breast milk, infant formula, growing-up milk.

The expression “mother's milk” should be understood as the breast milk or the colostrum of the mother.

Within the context of the present invention the term “ARA” or “AA” refers to C20:4n-6 (arachidonic acid).

Within the context of the present invention the term “DHA” refers to C22:6n-3 (docosahexaenoic acid).

The term “phospholipid” as used herein refers to any phospholipid, and in particular a compound of formula (I)

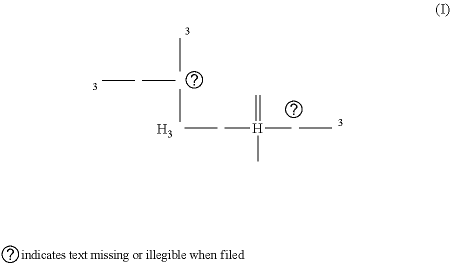

wherein,
R1 is O;
X is NH or O;
R2 is a C2-C44 saturated or unsaturated, linear or branched acyl group;
R3 is a substituent of formula (II) or (III):

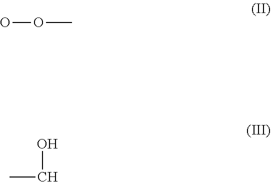

Wherein, R5 is a C2-C44 saturated or unsaturated, linear or branched acyl group and R6 is a C2-C44 saturated alkyl or alkenyl group; and
R4 is selected from: a C5 or C6 substituted or unsubstituted cyclic alkyl or alkenyl group, or
—(CH2)n-R7, wherein n is an integer ranging from 1 to 4, in particular 1 to 2 and R7 is —N(CH3)3+, or a substituent of formula (IV)

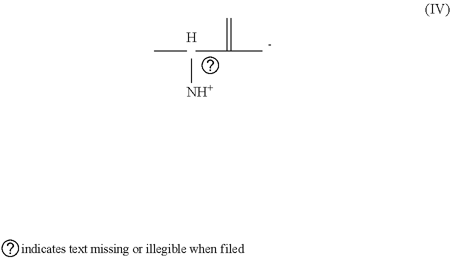

and;
in particular R4 is a C6 cyclic alkyl or alkyl or alkenyl group substituted with one or more hydroxy groups, more particularly R4 is derived from inositol (C6H12O6), and even more particularly myo-inositol i.e. R4 is:

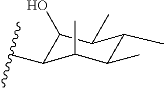 [SMILES: *C1C(C)C(C)C(C)C(C)C1O]

As used herein the term “acyclic” refers to a group that is not cyclic, i.e. does not contain a closed chain of atoms.

The term “phosphatidylinositol (PI)” refers to a compound of formula (V)

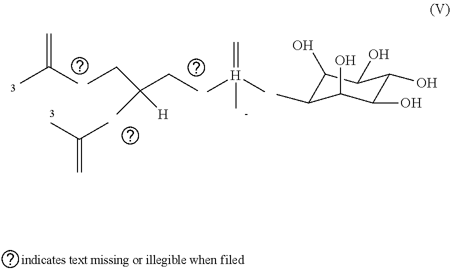

wherein R8 is a C2 to C43 branched or unbranched acyclic alkyl, or acyclic alkenyl group and,
R9 is a C2 to C43 branched or unbranched acyclic alkyl, or acyclic alkenyl group.

More particularly R8 and R9 are, independently of each other, C13 to C43 branched or unbranched acyclic alkyl, or acyclic alkenyl groups which together with their adjacent carbonyl group correspond to C14 to C44 saturated or unsaturated fatty acid residues, and even more particularly R8 and R9 are, independently of each other, branched or unbranched acyclic alkyl, or acyclic alkenyl group which together with their adjacent carbonyl group correspond to C14 to C24 saturated or unsaturated fatty acid residues.

More particularly R8 and R9 are C13 to C23 branched or unbranched acyclic alkyl, or acyclic alkenyl group which together with their adjacent carbonyl group are C14 to C24 saturated or unsaturated fatty residues, wherein the fatty acids from which the fatty acid residues stem are selected from the group consisting of: C14:0, C15:0, C16:0, C18:0, C20:0, C20:3, C20:4, C21:0, C22:0, C23:0, C24:0, C18:1n-9, C18:2n-6, and C24:1n-9, and even more particularly R8 and R9 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of: C18:0, C18:1n-9, C18:2, C20:3, and C20:4.

The term “phosphatidylserine (PS)” as used herein refers to phosphatidyl-L-Serine. Phosphatidylserine is a compound of formula (VI)

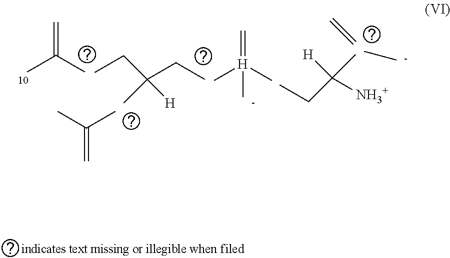

wherein R10 is a C2 to C43 branched or unbranched acyclic alkyl or acyclic alkenyl group and,
R11 is a C2 to C43 branched or unbranched acyclic alkyl or acyclic alkenyl group.

More particularly R10 and R11 are, independently of each other, C13 to C43 branched or unbranched acyclic alkyl, or acyclic alkenyl groups which together with their adjacent carbonyl group correspond to C14 to C44 saturated or unsaturated fatty acid residues, and even more particularly R10 and R11 are, independently of each other, C13 to C23 branched or unbranched acyclic alkyl or acyclic alkenyl groups which together with their adjacent carbonyl group correspond to C14 to C24 saturated fatty acid residues.

More particularly R10 and R11 are C13 to C23 branched or unbranched acyclic alkyl or acyclic alkenyl groups which together with their adjacent carbonyl group are C14 to C24 saturated or unsaturated fatty acid residues, wherein the fatty acids from which the fatty acid stem are selected from the group consisting of: C14:0, C15:0, C16:0, C18:0, C20:0, C20:3, C20:4, C21:0, C22:0, C23:0, C24:0, C18:1n-9, C18:2n-6, and C24:1n-9, and even more particularly R10 and R11 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of: C18:0, C18:1n-9, C18:2, C20:3, and C20:4.

The term “phosphatidylethanolamine (PE)” is a compound of formula (VII)

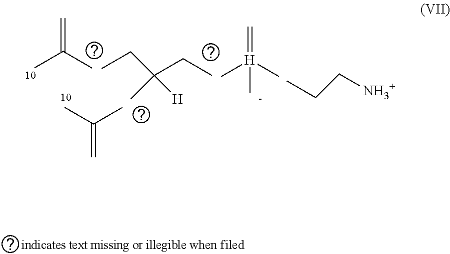

wherein R12 is a C2 to C43 branched or unbranched acyclic alkyl or acyclic alkenyl group and, R13 is a branched or unbranched acyclic alkyl or acyclic alkenyl group.

More particularly R12 and R13 are, independently of each other, C13 to C43 branched or unbranched acyclic alky or acyclic alkenyl groups which together with their adjacent carbonyl group correspond to C14 to C44 saturated or unsaturated fatty acid residues, and even more particularly R12 and R13 are, independently of each other, C13 to C23 branched or unbranched acyclic alkyl or acyclic alkenyl groups which together with their adjacent carbonyl group correspond to C14 to C24 saturated or unsaturated fatty acid residues.

The term “sphingomyelin (SM)” as used herein refers to a lipid molecule, or a mixture of lipid molecules, wherein a sphingosine or a shinganine backbone is esterified with a fatty acid residue at the amino group (—NH2) through an amide bound and wherein the hydroxyl group at position 1 of the sphingosine backbone is linked to a phophorylcholine moiety.

In a particular embodiment, sphingomyelin is a compound of formula (VIII) or a mixture of compounds of formula (VIII)

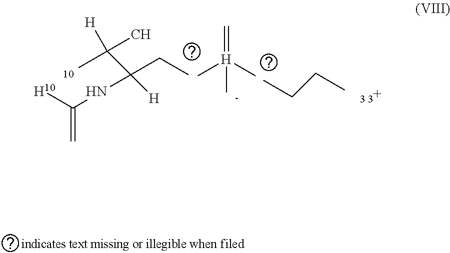

wherein R14 is a C2 to C43 branched or unbranched acyclic alkyl or acyclic alkenyl group, and R15 is a C2 to C43 branched or unbranched acyclic alkyl or acyclic alkenyl group.

More particularly R14 is a C13 to C43 branched or unbranched acyclic alkyl or acyclic alkenyl group which together with the adjacent carbonyl group correspond to a C14 to C44 saturated or unsaturated fatty acid residues.

Non-limiting examples of C14 to C44 saturated or unsaturated fatty acids from which the fatty acid residue may stem include: C14:0, C15:0, C16:0, C18:0, C20:0, C21:0, C22:0, C23:0, C24:1, C25:0, C28:1, C30:2, C30:1, C30:0, C32:3, C32:2, C32:1, C32:0, C33:1, C34:3, C34:2, C34:1, C34:0, C35:2, C35:0, C36:4, C36:3, C36:2, C36:1, C36:0, C37:1, C37:0, C38:4, C38:3, C38:1, C38:0, C39:1, C39:0, C40:2, C40:1, C40:0, C41:2, C41:1, C41:0, C42:47, C42:3, C42:2, C42:1, C42:0, C44:3, C44:1.

Even more particularly R14 is a C13 to C23 branched or unbranched acyclic alkyl or acyclic alkenyl group which together with the adjacent carbonyl group is a C14 to C24 saturated or unsaturated fatty acid residue, wherein the fatty acid from which the fatty acid residue may stem is selected from the group consisting of: C14:0, C15:0, C16:0, C18:0, C20:0, C21:0, C22:0, C23:0, C24:0, C18:1n-9, C18:2n-6, and C24:1n-9.

Even more particularly still, sphingomyelin is a mixture of compounds of formula (VIII) wherein the mixture is such that the total number of fatty acid residues (R14 together with the adjacent carbonyl group) comprised in the mixture are predominately saturated fatty acids, and the least predominant are unsaturated fatty acids. More particularly the mixture will be such that 80% to 96% of said fatty acid residues in the mixture are saturated fatty acids, in particular C14, C15, C16, C18, C20, C22, C23, C24 saturated fatty acids, more particularly C16, C18, C20, C22 and C24.

The term “phosphatidylcholine (PC)” refers to a compound of formula (IX)

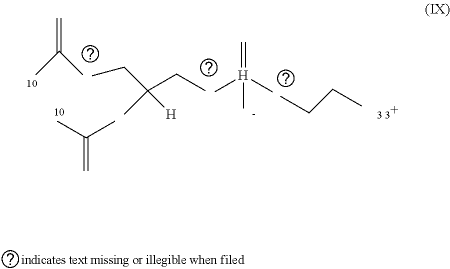

wherein R16 is a C2 to C43 branched or unbranched acyclic alkyl or acyclic alkenyl group and R17 is C2 to C43 branched or unbranched acyclic alkyl or acyclic alkenyl group.

More particularly, R16 and R17 are C13 to C23 C2 to C43 branched or unbranched acyclic alkyl or acyclic alkenyl groups which together with their adjacent carbonyl group correspond to C14 to C44 saturated or unsaturated fatty acid residues, end even more particularly R16 and R17 are, independently of each other, C13 to C23 branched or unbranched acyclic alky or acyclic alkenyl groups which together with their adjacent carbonyl group correspond to C14 to C24 saturated or unsaturated fatty acids residues.

More particularly, R16 and R17 are C13 to C23 branched or unbranched acyclic alky or acyclic alkenyl groups which together with their adjacent carbonyl group are C14 to C24 saturated or unsaturated fatty acid residues, wherein the fatty acid from which the fatty acid residues stem are selected from the group of: C14:0, C15:0, C16:0, C16:1, C18:0, C20:0, C20:1, C20:3, C20:4, C21:0, C22:0, C22:6, C23:0, C24:0, C18:1n-9, C18:2n-6, and C24:1n-9. Even more particularly C14:0, C16:0, C18:0, C18:1n-9, C18:2n-6, C20:1, C20:3, C20:4, and C22:6.

As used herein, “about” is understood to refer to numbers in a range of numerals, for example the range of −10% to +10% of the referenced number, within-5% to +5% of the referenced number, or in one aspect, within-1% to +1% of the referenced number, and in a specific aspect, within-0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

In the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

All percentages are by weight unless otherwise stated.

The terms “in particular” or “more particularly” as used herein should not be considered limiting but should be interpreted as being synonymous with “for example” or “especially”.

As used in this disclosure and the appended claims, the singular forms “a,” “an” and “the” include plural referents unless the context clearly dictates otherwise. Thus, for example, reference to “a component” or “the component” includes two or more components.

The objective of the present invention is hence to enrich or improve the state of the art and in particular to provide a combination of nutrients for use in improving social-emotional skills and/or promoting mood and joy behaviours in infants, toddlers and young children.

The publications discussed herein are provided solely for their disclosure prior to the filing date of the present application. Nothing herein is to be construed as an admission that such publications constitute prior art to the claims appended hereto.

### Embodiments of the Invention

The invention will be now described in further details.

In a first aspect, the present invention relates to a combination of DHA, ARA, iron, folic acid, Vitamin B12 and phospholipids for use in improving social-emotional skills and/or promoting mood and joy behaviour in an infant.

In one embodiment, the present invention provides a combination consisting of DHA, ARA, iron, folic acid, Vitamin B12 and phospholipids for use in improving social-emotional skills and/or in promoting mood and joy behaviour in an infant, a toddler or a young child.

In one embodiment, the combination according to the present invention comprises a phospholipid, a precursor or a metabolite thereof.

Non-limiting examples of phospholipids include phosphatidylinositol, phosphatidylserine, phosphatidylethanolamine, sphingomyelin and phosphatidylcholine.

In some embodiments of the present invention, the phospholipids are sphingomyelin, phosphatidylcholine, phosphatidylinositol, phosphatidylserine and phosphatidylethanolamine.

In some embodiments, the phospholipids comprise sphingomyelin, phosphatidylcholine, phosphatidylinositol, phosphatidylserine and phosphatidylethanolamine.

In other embodiments, phospholipids consist of sphingomyelin, phosphatidylcholine, phosphatidylinositol, phosphatidylserine and phosphatidylethanolamine.

The expression “total phospholipids” refers to the total amount of sphingomyelin, phosphatidylcholine, phosphatidylinositol, phosphatidylserine and phosphatidylethanolamine in the combination according to the present invention and/or in the nutritional composition.

In an embodiment of the present invention, the phospholipid comprises phosphatidylcholine, phosphatidylinositol, phosphatidylserine, phosphatidylethanolamine and sphingomyelin wherein sphingomyelin is comprised in an amount ranging from 20 to 30% w/w sphingomyelin based on total phospholipids.

In some embodiments of the present invention, the phospholipids comprise phosphatidylcholine, phosphatidylinositol, phosphatidylserine, phosphatidylethanolamine and sphingomyelin which are comprised at respective amounts ranging from 25 to 35% w/w phosphatidylcholine based on total phospholipids, amounts ranging from 5 to 15% w/w phosphatidylinositol on total phospholipids, amounts ranging from 5 to 10% w/w phosphatidylserine on total phospholipids, amounts ranging from 21 to 30% w/w phosphatidylethanolamine on total phospholipids and amounts ranging from 20 to 30% w/w sphingomyelin on total phospholipids.

Suitably, the phospholipids comprise phosphatidylcholine, phosphatidylinositol, phosphatidylserine, phosphatidylethanolamine and sphingomyelin which are comprised at respective amounts of 32% w/w phosphatidylcholine on total phospholipids, 11% w/w phosphatidylinositol on a total phospholipids, 8% w/w phosphatidylserine on a total phospholipids, 25% w/w phosphatidylethanolamine on total phospholipids and 24% w/w sphingomyelin on total phospholipids. In one embodiment of the present invention, the synthetic nutritional composition comprises total phospholipids in an amount of at least 150 mg/100 g of the synthetic nutritional composition. In one embodiment of the present invention, the synthetic nutritional composition comprises total phospholipids in an amount of at least 200 mg/100 g of the synthetic nutritional composition.

In one embodiment of the present invention, the synthetic nutritional composition comprises total phospholipids in an amount ranging from 200 to 400 mg/100 g of the synthetic nutritional composition.

In one embodiment, the synthetic nutritional composition comprises sphingomyelin in an amount higher than 200 mg/kg of the dry weight of the composition, more particularly ranging from 200 mg to 2.5 g/kg of the dry weight of the composition.

In an embodiment the synthetic nutritional composition comprises sphingomyelin in an amount selected from the group consisting of; higher than 200 mg/kg, higher than 300 mg/kg, ranging from 200 mg to 2.5 g/kg, ranging from 200 mg to 2 g/kg, in amount ranging from 300 mg to 1.5 g/kg or from 400 mg to 1 g/Kg, ranging from 200 to 850 mg/kg, or 300 to 820 mg/kg. All weights being per dry weight of the composition. In one embodiment, the composition will comprise phosphatidylcholine in an amount higher than 200 mg/kg of the dry weight of the composition, more particularly ranging from 200 mg to 2.5 g/kg of the dry weight of the composition.

In an embodiment the synthetic nutritional composition comprises phosphatidylcholine in an amount selected from the group consisting of; higher than 200 mg/kg, higher than 300 mg/kg, higher than 400 mg/kg, ranging from 200 mg to 2.5 g/kg, ranging from 200 mg to 2 g/kg, in amount ranging from 300 mg to 1.5 g/kg or from 400 mg to 1 g/Kg, 500 mg to 1.3 g/Kg. All weights being per dry weight of the composition.

In one embodiment, the synthetic nutritional composition will comprise phosphatidylinositol in an amount higher than 500 mg/kg of the dry weight of the composition, more particularly ranging from 200 mg to 1.5 g/kg of the dry weight of the composition.

In an embodiment the composition comprises phosphatidylinositol in an amount selected from the group consisting of; higher than 200 mg/kg, higher than 300 mg/kg, ranging from 200 mg to 2.5 g/kg, ranging from 200 mg to 2 g/kg, in amount ranging from 250 mg to 800 mg/kg or from 400 mg to 1.5 g/Kg, or from 400 to 800 mg/kg. All weights being per dry weight of the composition.

In one embodiment, the synthetic nutritional composition will comprise phosphatidylserine in an amount higher than 50 mg/kg of the dry weight of the composition, higher than 200 mg/kg of the dry weight of the composition, more particularly ranging from 150 mg to 1.5 g/kg of the dry weight of the composition, from 200 mg to 1 g/kg of the dry weight of the composition

In an embodiment the synthetic nutritional composition comprises phosphatidylserine in an amount selected from the group consisting of; higher than 150, higher than 200 mg/kg, higher than 300 mg/kg, ranging from 200 mg to 2.5 g/kg, ranging from 200 mg to 2 g/kg, in amount ranging from 200 mg to 1000 mg/kg or from 250 mg to 1 g/Kg. All weights being per dry weight of the composition.

In one embodiment, the composition will comprise phosphatidylethanolamine in an amount higher than 150 mg/kg of the dry weight of the composition, higher than 200 mg/kg of the dry weight of the composition, more particularly ranging from 150 mg to 1.5 g/kg of the dry weight of the composition.

In an embodiment the composition comprises phosphatidylethanolamine in an amount selected from the group consisting of; higher than 170 mg/kg, higher than 180 mg/kg, higher than 200 mg/kg, ranging from 200 mg to 2.5 g/kg, ranging from 200 mg to 2 g/kg, in amount ranging from 250 mg to 800 mg/kg or from 200 mg to 1 g/Kg. All weights being per dry weight of the composition.

If a metabolic precursor and/or metabolite of one or more phospholipid is used in a composition in place of or in combination with a phospholipid, said compounds may be used in amounts such that the level of phospholipids physiologically delivered by said composition is in line with those set out hereinabove. It is well within the purview of the skilled person to determine appropriate amounts.

The term metabolic precursor and/or metabolite of one or more phospholipid as used herein does not include choline.

The phospholipid, metabolic precursors and/or metabolite thereof, comprised in the composition of the invention may be natural, synthetic or a mixture thereof. Said metabolic precursors and/or a metabolite, may be used in the composition of the invention in their pure form, or substantially pure form. Alternatively, they may be added in the form of a source comprising them.

Any source of a phospholipid metabolic precursors and/or metabolite thereof, suitable for ingestion by a subject for which the composition is intended to be consumed may be used in the invention.

In particular, the phospholipid a metabolic precursor or metabolite thereof, will come from natural sources, non-limiting examples of which include, eggs, soy, bovine brains, and/or mammalian milk or extracts thereof. Non limiting examples of soy sources include soy lecithin-food additive, non-limiting examples of mammalian milk include bovine, camel, sheep, goat milk including skilled milks. Non limiting extracts of milk include protein extracts e.g. whey protein and casein, milk fat globule membranes (MFGM) and extracts comprising them.

A particularly useful source of a phospholipids a metabolic precursor or metabolite thereof, in particular sphingomyelin, that may be used in the present invention may be a bovine milk whey protein concentrate enriched in alpha-lactalbumin, and/or non-pure alpha-lactalbumin which has been extracted from milk whey protein, in particular bovine milk whey protein.

Alpha-Lactalbumin is a high-quality, easy-to-digest e.g. by human infants, whey protein and is the primary protein found in HM. Alpha-lactalbumin and/or an alpha-lactalbumin enriched milk fraction is ideal for use in lower protein infant formulas due to its high content of essential amino acids, particularly tryptophan. Although alpha-Lactalbumin is in itself a protein non pure sources may comprise sphingomyelin.

In an embodiment a phospholipid a metabolic precursor or metabolite thereof, in particular sphingomyelin, is used in the form of a whey protein concentrate enriched in alpha-lactalbumin or as alpha-lactalbumin.

In a more particular embodiment, a bovine whey protein concentrate enriched in alpha-lactalbumin or alpha-lactalbumin having a phospholipid content, in particular sphingomyelin content higher than 500 mg/100 g, 900 mg/100 g, 1000 mg/100 g dry weight of the composition is used.

Another particularly useful source of phospholipids a metabolic precursor, or metabolite thereof, may be milk fat globule membrane (hereinafter MFGM) or extracts comprising them, in particular MFGM, or extracts comprising them from bovine milk. It may be particularly beneficial if the MFGM or extracts comprising them comprises at least 1%, 2%, 5%, 10%, 20%, 30%, 40% phospholipids and/or at least 0.1%, 0.2%, 0.5% to 5%, 0.8% to 3%, 1% to 2%, 1.6%, 1.9%, 1.8% of phosphatidylcholine, phosphatidylinositol, phosphatidylserine, phosphatidylethanolamine, and/or sphingomyelin. The MFGM may also further comprise magnesium, phosphorus and or calcium, in particularly in concentrations ranging from 0.05% to 2%, 0.1% to 0.4%.

In an embodiment, the combination according to the present invention comprise Iron.

In particular, iron may be comprised in the nutritional composition in an amount higher than 5 mg/100 g of the dry composition.

In an embodiment, the nutritional composition according to the present invention comprise Iron in an amount selected from the group consisting of; higher than 4 mg, higher than 9 mg, ranging from 5 to 40 mg, ranging from 9 to 40 mg, ranging from 5 and 20 mg, ranging from 9 to 20 mg, ranging from 5 to 15 mg, ranging from 9 to 15 mg, ranging from 3.5 to 7 mg, wherein all weights are per 100 g of the dry composition.

Iron may be incorporated in the combination and/or nutritional compositions of the invention in the form of one physiologically acceptable salt such as, for example: ferric citrate, ferric phosphate, ferric pyrophosphate, ferrous ascorbate, ferrous carbonate, ferrous citrate, ferrous fumarate, ferrous gluconate, ferrous lactate, ferrous sulfate or mixtures thereof.

Iron may be incorporated in the combination and/or nutritional composition of the invention in the form of a physiologically acceptable iron complex (such as for example EDTA ferric sodium salt) or mixtures thereof.

Fe2+ is more bioavailable and it may therefore be more beneficial if iron is added into the composition in the form of a ferrous salt or complex e.g. a ferrous salts listed hereinabove.

In an embodiment, the nutritional composition according to the present invention comprises levels of iron such that the total daily intake derived from the nutritional composition of the invention will not exceed 40 mg.

In particular vitamin B12 may be comprised in the nutritional composition in an amount of selected from the group consisting of; higher than 0.01 mcg, in particular higher than 0.04 mcg, in particular higher than 0.05 mcg, wherein all weights are/100 g of the dry composition.

In an embodiment the nutritional composition of the invention comprises vitamin B12 in an amount selected from the group consisting of; higher than 0.01 mcg, higher than 0.5 mcg, higher than 0.7, higher than 5, ranging from 0.1 to 10 mcg, 0.4 to 5 mcg, 0.5 to 2 mcg, 1 to 1.5 mcg, 4 to 8.5 mcg, 5 to 8 mcg, wherein all weights are per 100 g of the dry composition.

In an embodiment, the nutritional composition according to the present invention comprises an amount of vitamin B12 such that the total daily intake derived from the nutritional composition of the invention will not exceed 7.6 mcg/100 g of the dry composition (77.6 mcg/Kg of the dry composition).

Vitamin B12 may be incorporated in the nutritional compositions of the invention as such or in the form of a physiologically acceptable salt thereof or mixtures thereof, or via any source comprising vitamin B12. In particular vitamin B12 may be incorporated into the composition in its pure form, as cyanocobalamin, hydroxocobalamin, and any combination thereof.

In particular folic acid may be comprised in an amount of higher than 50 mcg/100 g of the dry composition, more particularly 50 mcg to 500 mcg/100 g of the dry composition.

In an embodiment the nutritional composition of the invention comprises folic acid in an amount selected from the group consisting of; higher than 50 mcg, higher than 65 mcg, higher than 70 mcg, higher than 100 mcg, higher than 110 mcg, higher than 160 mcg, ranging from 50 to 500 mcg, ranging from 50 to 400 mcg, ranging from 70 to 170 mcg, ranging from 110 to 500 mcg, ranging from 110 to 400 mcg, ranging from 110 to 400 mcg, ranging from 110 to 350 mcg, wherein all weights are per 100 g of the dry composition.

In an embodiment, the nutritional composition according to the present invention comprises an amount of folic acid such that the total daily intake derived from the nutritional composition of the invention will not exceed 400 mcg.

Folic acid may be incorporated in the nutritional compositions of the invention as such or in the form of a physiologically acceptable salt thereof (folate) or mixtures thereof.

DHA and/or ARA may be comprised in the nutritional composition of the invention in an amount of 15 to 350 mg/100 g dry weight of the composition, more particularly 30 mg to 300 mg/100 g dry weight of the composition.

In an embodiment, the nutritional composition according to the present invention comprises DHA and/or ARA in an amount selected from the group consisting of; higher than 15 mg/100 g, higher than 30 mg/100 g, higher than 50 mg/100 g, higher than 55 mg/100 g, ranging from 30 to 300 mg/100 g, ranging from 30 to 200 mg/100 g or from 30 to 150 mg/100 g, ranging from 50 to 300 mg/100 g, ranging from 50 to 200 mg/100 g, ranging from 50 to 150 mg/100 g, ranging from 150 to 350, ranging from 60 to 350 mg/100 g, ranging from 60 to 120 mg/100 g, ranging from 100 to 110 mg/100 g. All concentrations are by dry weight of the composition.

Fatty acid derivatives comprising DHA and/or ARA are present in natural sources such as for example egg, algae, fungus or fish oil, algae, and in plants.

Oils comprising fatty acid derivatives comprising DHA and/or ARA and generally other polyunsaturated fatty acids (PUFAs), in particular EPA (eicosapentaenoic acid), may be of various origin. Preferably, fatty acid derivatives comprising DHA are provided in the form of a fish oil comprising fatty acid derivatives comprising DHA and/or ARA. Fish oils generally comprise 5 wt. % or more, preferably 10 wt. % or more of fatty acid derivatives comprising DHA and/or ARA. Oils comprising substantial amounts of fatty acid derivatives comprising DHA and/or ARA, obtained from algae or microorganisms in general are also available. For example, oils harvested from algae comprising 10 wt. % or more, for example 20 wt. % or more of fatty acid derivatives, may be used.

ARA and DHA may for example be comprised in the composition of the invention in amounts resulting in a weight ratio of DHA:ARA in the range of 4:1 to 1:4, for example 3:1 to 1:3, for example 2:1 to 1:2, for example 1.5:1 to 1:1.5, in particular 1.1:1 to 1:1.1.

Further, when high amounts of fatty acid derivatives comprising DHA and/or ARA are comprised in the composition of the invention, it may be particularly beneficial if the total amount of fatty acid derivatives comprising saturated long chain fatty acids, in particular C20/24 is increased. These saturated long chain fatty acids may be an important component of myelin enabling it to wrap around and enrobe axons. The weight ratio of DHA and/or AA to these unsaturated long fatty acids in the composition of the invention may for example be within the range 1:1 1:10; 1:2 to 1:9, 1:3 to 1:4.5, 1:3.5 to 1:4.5.

The nutritional composition according to the invention can be for example an infant formula, a starter infant formula, a follow-on or follow-up formula, a baby food, an infant cereal composition, a fortifier such as a human milk fortifier, or a supplement.

In an embodiment, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milk fortifier) or a formula fortifier such as an infant formula fortifier or a follow-on/follow-up formula fortifier.

When the nutritional composition is a supplement, it can be provided in the form of unit doses.

The nutritional composition of the present invention can be in solid (e.g. powder), liquid or gelatinous form.

In one embodiment of the present invention, the nutritional composition is a synthetic nutritional composition.

In one embodiment of the present invention a synthetic nutritional composition is provided which comprises sphingomyelin in an amount ranging from 300 to 820 mg/kg, phosphatidylcholine in an amount 500 mg/Kg to 1.3 g/Kg, phosphatidylinositol in an amount 400 mg/Kg to 800 mg/kg, phosphatidylserine in an amount ranging from 200 mg/Kg to 1000 mg/kg, phosphatidylethanolamine in an amount ranging from 250 mg/Kg to 800 mg/kg, iron in an amount ranging from ranging from 3.5 mg/100 g to 7 mg/100 g, Vitamin B12 in an amount ranging from ranging from 0.1 mcg/100 g to 10 mcg/100 g, folic acid in an amount ranging from ranging from 110 mcg/100 g to 400 mcg/100 g and DHA and ARA in an amount 30 mg/100 g to 300 mg/100 g.

In some embodiments, the combination and/or the nutritional is administered during the first 1, 3, 6, 9, 12, 18, 14, or 36 months of life of a subject. For example, the combination and/or the nutritional is administered to a subject starting from birth up to 12, 18, 24 or 36 months.

The combination and/or the nutritional composition according to the present invention advantageously improves social-emotional skills. Particularly, the combination and/or the nutritional composition of the present invention improves in particular for improving behaviours in social interactions, for example play, and/or in response to novelty and/or new situations, and/or with new people and/or strangers in an infant older than 12 months, a toddler or a young child. Improving social-emotional skills in an infant, a toddler and/or a young child may reduce the risk of developing behavioural problems later in life, for example anxiety disorder, as it is believed that lack of social skills can lead in poor outcomes in child development such as hindering child's ability to create relationship, affecting child's adjustment at school, leading to loneliness and may cause behavioral problems later in life.

In some embodiments, the combination and/or nutritional composition improves social-emotional skills of a subject when administered to a subject during the first 1, 3, 6, 9, 12, 18, 14, or 36 months of life.

Suitably, the combination and/or nutritional composition according to the present invention improves social-emotional skills of a subject at 12, 18, 24 or 36 months of age. In one embodiment, the combination and/or nutritional composition according to the present invention improves social-emotional skills of a subject aged between 12 to 36 months. In another embodiment, combination and/or nutritional composition according to the present invention improves social-emotional skills of a subject of at least 12 months of age, of at least 18 months of age, or of at least 24 months of age.

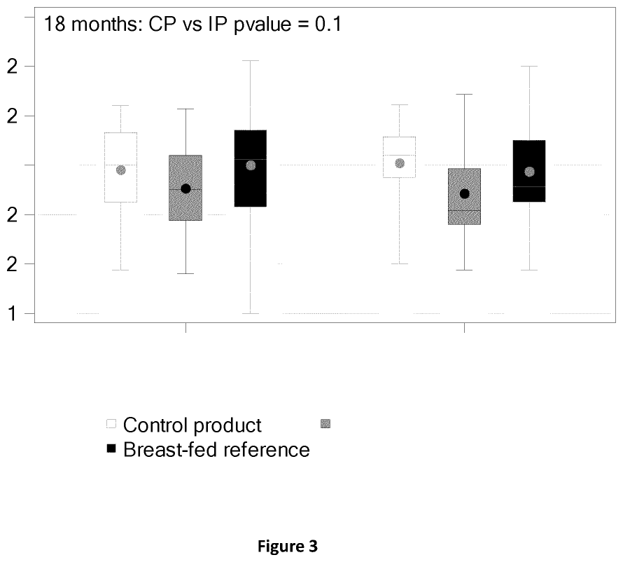

The inventors have surprisingly found that the composition and/or the nutritional composition according to the present invention improves social fear (FIG. 3 and Examples), in particular it improves inhibited approaches and/or distress in social situations that involves novelty or uncertainty in an infant older than 12 months, a toddler or a young child. In one embodiment, the combination and/or nutritional composition according to the present invention improves social fear when administered to a subject during the first 1, 3, 6, 9, 12, 18, 14, or 36 months of life.

In one embodiment, the composition and/or the nutritional composition according to the present invention is for use for improving social-emotional skills in an infant, a toddler or a young child.

In some embodiments of the present invention, the combination and/or nutritional composition promotes high intensity pleasure, in particular pleasure and enjoyment related to high stimulus intensity, rate, complexity, novelty and incongruity, and reduces sadness, in particular reducing lowered mood and activity related to suffering physical state, object loss or inability to perform an action in an infant. Promoting mood and joy behaviors in an infant may reduce the risk of experiencing mood difficulties that may lead to aggressive or disruptive behaviors, to loneliness and may cause behavioral problems later in life.

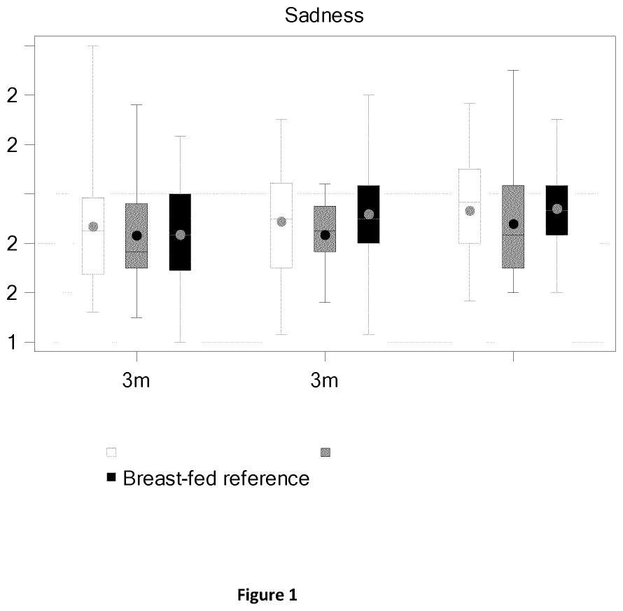

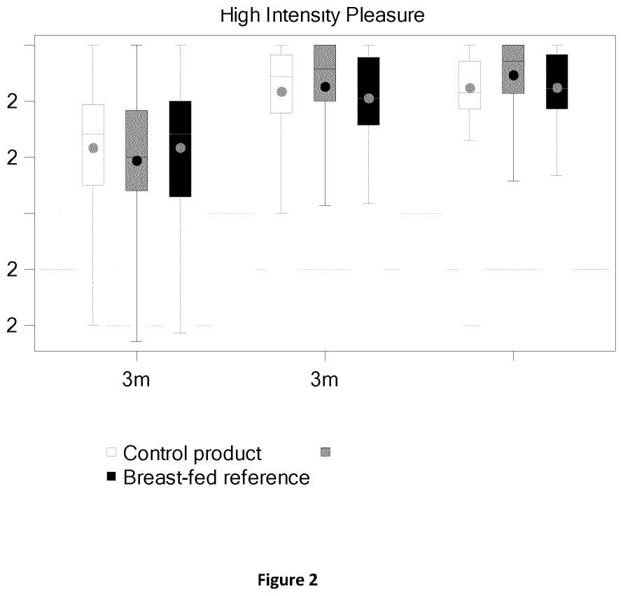

The inventors have surprisingly found that the combination and/or nutritional composition according to the present invention reduces sadness (FIG. 1 and Examples) and promotes high intensity pleasure (FIG. 2 and Examples) in infants up to 12 months old.

In some embodiments, the combination and/or nutritional composition promotes mood and joy behaviors in a subject between the age of 0 and 18 months, preferably between the age of 0 to 12 months.

In some embodiments, the composition and/or the nutritional composition according to the present invention is for use for promoting mood and joy behaviors.

In some embodiments, the nutritional composition is a synthetic nutritional composition.

Another aspect of the present invention relates to a synthetic nutritional composition for use in improving social-emotional skills and/or for promoting mood and joy behaviors in an infant, a toddler or a young child, said synthetic nutritional composition comprising a combination of DHA, ARA, iron, folic acid, Vitamin B12 and phospholipids as described above.

Suitably, the synthetic nutritional composition may be for example an infant formula, a starter infant formula, a follow-on or follow-up infant formula, a baby food, a growing-up milk, an infant cereal composition, a fortifier or a supplement. In one embodiment, the nutritional composition is an infant formula, a fortifier or a supplement that may be intended for the first 3 to 12 months of age.

In another aspect, the present invention relates to the non-therapeutical use of a combination or nutritional composition as described above for improving social-emotional skills and/or promoting mood and joy behaviours in an infant, a toddler or a young child.

The present invention further relates to a method for improving social-emotional skills and/or promoting mood and joy behaviours in an infant, a toddler or a young child, said method comprising administering to the infant, the toddler or the young child a combination or a nutritional composition DHA, ARA, iron, folic acid, Vitamin B12 and phospholipids as described above.

In a last aspect, the present invention relates to the use of a combination of DHA, ARA, iron, folic acid, Vitamin B12 and phospholipids as described above in the manufacture of a nutritional composition for improving social-emotional skills and/or for promoting mood and joy behaviors on an infant, a toddler or a young child.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

The inventors investigated the effect of a nutrient blend of docosahexaenoic acid, arachidonic acid, iron, vitamin B12, folic acid as well as sphingomyelin from a uniquely proceed whey protein concentrate enriched in alpha-lactalbumin and phospholipids on infant and toddler behavior.

### Material and Methods

N=81 enrolled full term, neurotypical infants of both sexes were randomized into either the investigational (N=42) or the control group (N=39). N=108 breast-fed infants served as a reference group.

A nutritional composition according to the present invention (i.e an infant formula comprising a blend of docosahexaenoic acid, arachidonic acid, iron, vitamin B12, folic acid and sphingomyelin from a uniquely processed whey protein concentrate enriched in alpha-lactalbumin and phospholipids (as reported in Table 2 below).

Intervention products were bovine milk-based routine infant formulas manufactured by Wyeth Nutrition, Askeaton, Ireland. The alpha-lactalbumin enriched whey protein concentrate used for the control product was almost devoid of phospholipids and sphingomyelin, while the alpha-lactalbumin enriched whey protein concentrate used in the investigational product contained higher levels of sphingomyelin and phospholipids as a result of this ingredient's unique manufacturing process. The investigational product also contained higher levels of DHA, ARA, iron (fortified through Ferrous Sulfate Heptahydrate), folic acid and vitamin B12 (fortified through Cyanocobalamine) (Table 2 and 3) than the control product.

Infant and toddler behaviors were assessed with the respective age-appropriate version of a behavior questionnaire (IBQ-R up to 12 months and TBAQ for 18 and 24 months).

Both intervention groups were formally compared using descriptive statistics, independent t-tests as well as ANCOVA corrected for stratification factors. The p-values were not corrected for multiplicity.

TABLE 2
Nutrient levels in study intervention groups (nutritional composition
of intervention for investigational product (IP) (according to
the invention) and control product (CP)) after reconstitution
and breastfeeding (BF) (non-randomized reference group)

| | Investigational | Control |
|---|---|---|
| | Levels | Levels |
| Energy (kcal per | 132 | 132 |
| 200 ml serving) | | |
| Sphingomyelin (mg/L) | 84 | 22 |
| DHA (mg/L) | 128 | 71 |
| ARA (mg/L) | 132 | 71 |
| Iron (mg/L) | 6.9 | 4.0 |
| Folic Acid (mcg/L) | 175 | 85 |
| B12 (mcg/L) | 5.6 | 0.9 |
| Phospholipids (mg/L) | 410 | 186 |

TABLE 3
Nutrient levels in study intervention groups (nutritional
composition of intervention for investigational product (IP)
(according to the invention) and control product (CP))

| | Investigational | Control |
|---|---|---|
| | Levels | Levels |
| Sphingomyelin (mg/100 g) | 65 | 17 |
| DHA (mg/100 g) | 99 | 55 |
| ARA (mg/100 g) | 102 | 55 |
| Iron (mg/100 g) | 5.3 | 3.1 |
| Folic Acid (mcg/100 g) | 136 | 65.5 |
| B12 (mcg/100 g) | 4.32 | 0.71 |
| Phospholipids (mg/100 g) | 318 | 144 |

In the nutritional composition according to the invention described sphingomyelin was comprised in an amount ranging from 300 mg/Kg to 820 mg/kg, phosphatidylcholine was comprised in an amount 500 mg to 1.3 g/Kg, phosphatidylinositole was comprised in an amount 400 mg/kg to 800 mg/kg, phosphatidylserine was comprised in an amount ranging from 200 mg/kg to 1000 mg/kg and phosphatidylethanolamine was comprised in an amount ranging from 250 mg/kg to 800 mg/kg.

### Results

Intention to treat analyses included N=39 children in the investigational and N=42 in the control group at 24 months. Children receiving the investigational nutrient blend showed significantly reduced social fear at 24 months (p=0.046) (FIG. 3), a trend towards improved high intensity pleasure at 12 months (score 6.5 vs 6.2) (FIG. 2) and a trend towards reduced sadness at 3 months (score 3.4 vs 3.7) (FIG. 1) in favor of the investigational group.

### Discussion

Our findings support the impact of a myelin-specific nutrient blend on infant and toddler behavior, more specifically on aspects of social and emotional development, in term-born, well-nourished children. This adds important knowledge in a population where nutritional intervention studies are still scarce. Social fear, an aspect of sociability linked to hesitation, distress, or shyness in novel social situations like meeting strangers, is part of normal development. Increased social fear during toddlerhood has, however, been associated with higher risk for anxiety behaviors in later childhood.

### EMBODIMENTS

Various preferred features and embodiments of the present invention will now be described with reference to the following paragraphs (para).
1. A combination of DHA, ARA, iron, folic acid, Vitamin B12 and phospholipids for use in improving social-emotional skills and/or promoting mood and joy behaviours in an infant, a toddler or a young child.
2. A combination for use according to para 1, wherein the phospholipids are sphingomyelin, phosphatidylcholine, phosphatidylinositol, phosphatidylserine and phosphatidylethanolamine.
3. A combination for use according to paragraphs 1 or 2 wherein the phospholipids are phosphatidylcholine, phosphatidylinositol, phosphatidylserine, phosphatidylethanolamine and sphingomyelin and wherein such phospholipids are comprised at respective amounts ranging from 25 to 35% w/w phosphatidylcholine based on total phospholipids, amounts ranging from 5 to 15% w/w phosphatidylinositol on total phospholipids, amounts ranging from 5 to 10% 10 w/w phosphatidylserine on total phospholipids, amounts ranging from 21 to 30% w/w phosphatidylethanolamine on total phospholipids and amounts ranging from 20 to 30% w/w sphingomyelin on total phospholipids.
4. A combination for use according to anyone of para 1-3 wherein the phospholipids are phosphatidylcholine, phosphatidylinositol, phosphatidylserine, phosphatidylethanolamine and sphingomyelin and wherein such phospholipids are comprised at respective amounts of 32% w/w phosphatidylcholine on total phospholipids, 11% w/w phosphatidylinositol on a total phospholipids, 8% w/w phosphatidylserine on a total phospholipids, 25% w/w phosphatidylethanolamine on total phospholipids and 24% w/w sphingomyelin on total phospholipids.
5. A combination for use according to anyone of para 1-4 for improving social-emotional skills, in particular for improving behaviours in social interactions, for example play, and/or in response to novelty and/or new situations and/or with new people/strangers in an infant older than 12 months, a toddler or a young child.
6. A combination for use according to anyone of para 1-4 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
7. A combination for use according to anyone of para 1-6 for improving social fear, in particular for improving inhibited approaches and/or distress in social situations that involves novelty/uncertainty in an infant older than 12 months, a toddler or a young child.
8. A combination for use according to anyone of para 1-7 when said combination is administered during the first 1, 3, 6, 9, 12, 18, 24, 36 months of life of the subject.
9. A combination for use according to anyone of para 1-8, wherein social skills are improved in a subject at 12, 18, 24, 36 months.
10. A combination for use according to anyone of para 1-8, wherein mood and joy behaviours are promoted in a subject between 0 and 18 months of age, preferably between 0 and 12 months of age.
11. A nutritional composition for use in improving social-emotional skills and promoting mood and joy behaviours in an infant, a toddler or a young child which comprises a combination of DHA, ARA, iron, folic acid, Vitamin B12 and phospholipids as described in anyone of paragraphs 1 to 10.
12. A nutritional composition for use according to para 11 which is a synthetic nutritional composition.
13. A synthetic nutritional composition for use according to anyone of para 11-12, wherein said synthetic nutritional composition is an infant formula, a starter infant formula, a follow-on or follow-up infant formula, a baby food, a growing-up milk, an infant cereal composition, a fortifier or a supplement.
14. A synthetic nutritional composition for use according to anyone of para 11-13 which comprises sphingomyelin in an amount ranging from 300 mg/kg to 820 mg/kg, phosphatidylcholine in an amount ranging from 500 mg/kg to 1.3 g/kg, phosphatidylinositol in an amount ranging from 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from 3.5 mg/100 g to 7 mg/100 g, Vitamin B12 in an amount ranging from 0.1 mcg/100 g to 10 mcg/100, folic acid in an amount ranging from 110 mcg/100 g to 400 mcg/100 g and DHA and ARA in an amount ranging from 30 mg/100 g to 300 mg/100 g.
15. A nutritional composition for use according to anyone of para 11-14 for improving social-emotional skills, in particular for improving behaviours in social interactions, for example play, and/or in response to novelty and/or new situations and/or with new people/strangers in an infant older than 12 months, a toddler or a young child.
16. A nutritional composition for use according to anyone of para 11-14 for promoting high intensity pleasure, in particular pleasure and enjoyment related to high stimulus intensity, rate, complexity, novelty and incongruity, and for reducing sadness, in particular reducing lowered mood and activity related to suffering physical state, object loss or inability to perform desired action in an infant.
17. A nutritional composition for use according to anyone of para 11-15 for improving social fear, in particular for improving inhibited approaches and/or distress in social situations that involves novelty/uncertainty in an infant older than 12 months, a toddler or a young child.
18. A nutritional composition for use according to anyone of para 11-17 wherein said nutritional composition is administered during the first 1, 3, 6, 9, 12, 18, 24, 36 months of life of the subject.
19. A nutritional composition for use according to anyone of para 11-18 wherein social-emotional skills are improved in a subject at 12, 18, 24, 36 months.
20. A nutritional composition for use according to anyone of para 11-18, wherein mood and joy behaviours are promoted in a subject between 0 and 18 months of age, preferably between 0 and 12 months of age.
21. A combination of DHA, ARA, iron, folic acid, Vitamin B12 and phospholipids, wherein the phospholipids are sphingomyelin, phosphatidylcholine, phosphatidylinositol, phosphatidylserine and phosphatidylethanolamine and wherein phospholipids are comprised in respective amounts ranging from 25 to 35% w/w phosphatidylcholine based on total phospholipids, amounts ranging from 5 to 15% w/w phosphatidylinositol on total phospholipids, amounts ranging from 5 to 10% w/w phosphatidylserine on total phospholipids, amounts ranging from 21 to 30% w/w phosphatidylethanolamine on total phospholipids and amounts ranging from 20 to 30% w/w sphingomyelin on total phospholipids.
22. Non-therapeutical use of a combination according to para 21 for improving social-emotional skills and/or for promoting mood and joy behaviours in an infant, a toddler or a young child.
23. Non-therapeutical use of a combination according to anyone of para 21-22 for improving social-emotional skills, in particular for improving behaviours in social interactions, for example play, and/or in response to novelty and/or new situations and/or with new people/strangers in an infant older than 12 months, a toddler or a young child.
24. Non-therapeutical use of a combination according to anyone of para 21-23 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
25. Non-therapeutical use of a combination according to anyone of para 12-24 for improving social fear, in particular for improving inhibited approaches and/or distress in social situations that involves novelty/uncertainty in an infant older than 12 months, a toddler or a young child.
26. Non-therapeutical use of a combination according to anyone of para 21-25, wherein said nutritional composition is administered during the first 1, 3, 6, 9, 12, 18, 24, 36 months of life of the subject.
27. Non-therapeutical use of a combination according to anyone of para 21-26, wherein social-emotional skills are improved in a subject at 12, 18, 24, 36 months.
28. Non-therapeutical use of a combination according to anyone of para 21-27, wherein mood and joy behaviours are promoted in a subject between 0 and 18 months of age, preferably between 0 and 12 months of age.
29. A nutritional composition which comprises a combination according to para 21.
30. A nutritional composition according to para 29 which is a synthetic nutritional composition.
31. Non-therapeutical use of a nutritional composition according to anyone of para 29-30 for improving social-emotional skills and/or for promoting mood and joy behaviours in an infant, a toddler or a young child.
32. Non-therapeutical use of a nutritional composition according to anyone of para 29-31 for improving social-emotional skills, in particular for improving behaviours in social interactions, for example play, and/or in response to novelty and/or new situations and/or with new people/strangers in an infant older than 12 months, a toddler or a young child.
33. Non-therapeutical use of a nutritional composition according to anyone of para 29-31 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
34. Non-therapeutical use of a nutritional composition according to anyone of para 29-33 for improving social fear, in particular for improving inhibited approaches and/or distress in social situations that involves novelty/uncertainty in an infant older than 12 months, a toddler or a young child.
35. Non-therapeutical use of a nutritional composition according to anyone of para 29-34 wherein said nutritional composition is administered during the first 1, 3, 6, 9, 12, 18, 24, 36 months of life of the subject.
36. Non-therapeutical use of a nutritional composition according to anyone of para 29-35, wherein social-emotional skills are promoted in a subject at 12, 18, 24, 36 months.
37. Non-therapeutical use of a nutritional composition according to anyone of para 29-36, wherein mood and joy behaviours are promoted in a subject between 0 and 18 months of age, preferably between 0 and 12 months of age.
38. A synthetic nutritional composition according to anyone of para 30 which comprises sphingomyelin in an amount ranging from 300 mg/kg to 820 mg/kg, phosphatidylcholine in an amount ranging from 500 mg/kg to 1.3 g/kg, phosphatidylinositol in an amount ranging from 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from 3.5 mg/100 g to 7 mg/100 g, Vitamin B12 in an amount ranging from 0.1 mcg/100 g to 10 mcg/100, folic acid in an amount ranging from 110 mcg/100 g to 400 mcg/100 g and DHA and ARA in an amount ranging from 30 mg/100 g to 300 mg/100 g.
39. Non-therapeutical use of a synthetic nutritional composition according to anyone of 30 or 37 for improving social-emotional skills and/or for promoting mood and joy behaviours in an infant, a toddler or a young child.
40. Non-therapeutical use of a synthetic nutritional composition according to anyone of para 37-39, wherein said synthetic nutritional composition is an infant formula, a starter infant formula, a follow-on or follow-up infant formula, a baby food, a growing-up milk, an infant cereal composition, a fortifier or a supplement.
41. Non-therapeutical use of a synthetic nutritional composition according to anyone of para 37-40 which comprises sphingomyelin in an amount ranging from 300 mg/kg to 820 mg/kg, phosphatidylcholine in an amount ranging from 500 mg/kg to 1.3 g/kg, phosphatidylinositol in an amount ranging from 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from 3.5 mg/100 g to 7 mg/100 g, Vitamin B12 in an amount ranging from 0.1 mcg/100 g to 10 mcg/100, folic acid in an amount ranging from 110 mcg/100 g to 400 mcg/100 g and DHA and ARA in an amount ranging from 30 mg/100 g to 300 mg/100 g.
42. Non-therapeutical use of a synthetic nutritional composition according to anyone of para 37-41 for improving social-emotional skills, in particular for improving behaviours in social interactions, for example play, and/or in response to novelty and/or new situations and/or with new people/strangers in an infant older than 12 months, a toddler or a young child.
43. Non-therapeutical use of a synthetic nutritional composition according to anyone of para 37-42 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
44. Non-therapeutical use of a synthetic nutritional composition according to anyone of para 37-43 for improving social fear, in particular for improving inhibited approaches and/or distress in social situations that involves novelty/uncertainty in an infant older than 12 months, a toddler or a young child.
45. Non-therapeutical use of a synthetic composition according to anyone of para 37-44 wherein said synthetic nutritional composition is administered during the first 1, 3, 6, 9, 12, 18, 24, 36 months of life of the subject.
46. Non-therapeutical use of a synthetic composition according to anyone of para 37-45 wherein social-emotional skills are promoted in a subject at 12, 18, 24, 36 months.
47. Non-therapeutical use of a synthetic composition according to any one of para 37-46 wherein mood and joy behaviours are promoted in a subject between 0 and 18 months of age, preferably between 0 and 12 months of age.
48. A method for improving social-emotional skills and/or for promoting mood and joy behaviours in an infant, a toddler or a young child, the method comprising administering to said infant, toddler or young child an effective amount of a combination of DHA, ARA, iron, folic acid, Vitamin B12 and phospholipids.
49. Method according to para 48, wherein the phospholipids are sphingomyelin, phosphatidylcholine, phosphatidylinositol, phosphatidylserine and phosphatidylethanolamine.
50. Method according to anyone of para 48-49, wherein the phospholipids are phosphatidylcholine, phosphatidylinositol, phosphatidylserine, phosphatidylethanolamine and sphingomyelin and wherein such phospholipids are comprised at respective amounts ranging from 25 to 35% w/w phosphatidylcholine based on total phospholipids, amounts ranging from 5 to 15% w/w phosphatidylinositol on total phospholipids, amounts ranging from 5 to 10% w/w phosphatidylserine on total phospholipids, amounts ranging from 21 to 30% w/w phosphatidylethanolamine on total phospholipids and amounts ranging from 20 to 30% w/w sphingomyelin on total phospholipids.
51. Method according to anyone of para 48-50 for improving social-emotional skills, in particular for improving behaviours in social interactions, for example play, and/or in response to novelty and/or new situations and/or with new people/strangers in an infant older than 12 months, a toddler or a young child.
52. Method according to anyone of para 48-51 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
53. Method according to anyone of para 48-51 for improving social fear, in particular for improving inhibited approaches and/or distress in social situations that involves novelty/uncertainty in an infant older than 12 months, a toddler or a young child.
54. A method according to anyone of para 48-53 wherein the effective amount of the combination is administered during the first 1, 3, 6, 9, 12, 18, 24, 36 months of life of the subject.
55. A method according to anyone of para 48-54 wherein social skills are improved in a subject at 12, 18, 24, 36 months.
56. A method according to anyone of para 48-54 wherein mood and joy behaviours are promoted in a subject between 0 and 18 months of age, preferably between 0 and 12 months of age.
57. A method for improving social-emotional skills and/or for promoting mood and joy behaviours in an infant, a toddler or a young child in an infant, toddler or young child, the method comprising administering to said infant, a toddler or a young child a nutritional composition which comprises a combination of DHA, ARA, iron, folic acid, Vitamin B12 and phospholipids.
58. A method according to para 57, wherein the phospholipids are sphingomyelin, phosphatidylcholine, phosphatidylinositol, phosphatidylserine and phosphatidylethanolamine.
59. A method according to anyone of para 57-58, wherein the phospholipids are phosphatidylcholine, phosphatidylinositol, phosphatidylserine, phosphatidylethanolamine and sphingomyelin and wherein such phospholipids are comprised at respective amounts ranging from 25 to 35% w/w phosphatidylcholine based on total phospholipids, amounts ranging from 5 to 15% w/w phosphatidylinositol on total phospholipids, amounts ranging from 5 to 10% w/w phosphatidylserine on total phospholipids, amounts ranging from 21 to 30% w/w phosphatidylethanolamine on total phospholipids and amounts ranging from 20 to 30% w/w sphingomyelin on total phospholipids.
60. A method according to anyone of para 57-59, for improving social-emotional skills, in particular for improving behaviours in social interactions, for example play, and/or in response to novelty and/or new situations and/or with new people/strangers in an infant older than 12 months, a toddler or a young child.
61. A method according to anyone of para 57-59 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
62. A method according to anyone of para 57-61 for improving social fear, in particular for improving inhibited approaches and/or distress in social situations that involves novelty/uncertainty in an infant older than 12 months, a toddler or a
63. A method according to anyone of para 57-62 wherein the nutritional composition is administered during the first 1, 3, 6, 9, 12, 18, 24, 36 months of life of the subject.
64. A method according to anyone of para 57-63 wherein social-emotional skills are promoted in a subject at 12, 18, 24, 36 months.
65. A method according to anyone of para 57-63 wherein mood and joy behaviours are promoted in a subject between 0 and 18 months of age, preferably between 0 and 12 months of age.
66. A method according to anyone of para 57-65 wherein the nutritional composition is a synthetic nutritional composition.
67. A method according to para 66, wherein the synthetic nutritional composition comprises sphingomyelin in an amount ranging from 300 mg/kg to 820 mg/kg, phosphatidylcholine in an amount ranging from 500 mg/kg to 1.3 g/kg, phosphatidylinositol in an amount ranging from 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from 3.5 mg/100 g to 7 mg/100 g, Vitamin B12 in an amount ranging from 0.1 mcg/100 g to 10 mcg/100, folic acid in an amount ranging from 110 mcg/100 g to 400 mcg/100 g and DHA and ARA in an amount ranging from 30 mg/100 g to 300 mg/100 g.
68. Use of a combination of DHA, ARA, iron, folic acid, Vitamin B12 and phospholipids in the manufacture of a nutritional composition for improving social-emotional skills and/or for promoting mood and joy behaviours in an infant, a toddler or a young child.
69. Use of a combination according to para 68, wherein the phospholipids are sphingomyelin, phosphatidylcholine, phosphatidylinositol, phosphatidylserine and phosphatidylethanolamine.
70. Use of a combination according to anyone of para 68-69, wherein the phospholipids are sphingomyelin, phosphatidylcholine, phosphatidylinositol, phosphatidylserine and phosphatidylethanolamine and wherein phospholipids are comprised in respective amounts ranging from 25 to 35% w/w phosphatidylcholine based on total phospholipids, amounts ranging from 5 to 15% w/w phosphatidylinositol on total phospholipids, amounts ranging from 5 to 10% w/w phosphatidylserine on total phospholipids, amounts ranging from 21 to 30% w/w phosphatidylethanolamine on total phospholipids and amounts ranging from 20 to 30% w/w sphingomyelin on total phospholipids.
71. Use of a combination according to anyone of para 68-70 for improving social-emotional skills, in particular for improving behaviours in social interactions, for example play, and/or in response to novelty and/or new situations and/or with new people/strangers in an infant older than 12 months, a toddler or a young child.
72. Use of a combination according to anyone of para 68-70 for promoting high intensity pleasure, in particular pleasure or enjoyment related to high stimulus intensity, rate, complexity, novelty and incongruity, and for reducing sadness, in particular reducing lowered mood and activity related to suffering, physical state, object loss or inability to perform desired action in an infant.
73. Use of a combination according to anyone of para 68-72-1 improving social fear, in particular for improving inhibited approaches and/or distress in social situations that involves novelty/uncertainty in an infant older than 12 months, a toddler or a
74. Use of a combination according to anyone of para 68-73 wherein the combination is administered during the first 1, 3, 6, 9, 12, 18, 24, 36 months of life of the subject.
75. Use of a combination according to anyone of para 68-74 wherein social skills are improved in a subject at 12, 18, 24, 36 months.
76. Use of a combination according to anyone of para 68-74 wherein mood and joy behaviours are promoted in a subject between 0 and 18 months of age, preferably between 0 and 12 months of age.

1: A method of improving social-emotional skills and/or in promoting mood and joy behaviour in an infant, a toddler or a young child, the method comprising administering a combination of DHA, ARA, iron, folic acid, Vitamin B12 and phospholipids to the infant, toddler or young child.

2: The method according to claim 1, wherein the phospholipids are sphingomyelin, phosphatidylcholine, phosphatidylinositol, phosphatidylserine and phosphatidylethanolamine.

3: The method according to claim 1 wherein the phospholipids are phosphatidylcholine, phosphatidylinositol, phosphatidylserine, phosphatidylethanolamine and sphingomyelin, and wherein the phospholipids are comprised at respective amounts ranging from 25 to 35% w/w of the phosphatidylcholine based on total phospholipids, amounts ranging from 5 to 15% w/w of the phosphatidylinositol based on total phospholipids, amounts ranging from 5 to 10% w/w of the phosphatidylserine based on total phospholipids, amounts ranging from 21 to 30% w/w of the phosphatidylethanolamine based on total phospholipids and amounts ranging from 20 to 30% w/w of the sphingomyelin based on total phospholipids.

4: The method according to claim 1 for improving inhibited approaches and/or distress in social situations that involves novelty/uncertainty in the infant, toddler or young child.

5: The method according to claim 1 for promoting high intensity pleasure sadness.

6: The method according to claim 1 wherein the combination is administered during the first month of life of the infant, toddler or young child.

7: The method according to claim 1 wherein social-emotional skills are improved in the infant, toddler or young child at the age of 12, 18, 24 or 36 months.

8: The method according to claim 1 anyone of the preceding claims wherein mood and joy behaviours are promoted in the infant, toddler or young child between the age of 0 to 18 months.

9: The method of claim 1, wherein the combination is administered in a nutritional composition.

10: The method of claim 1, wherein the combination is administered in a synthetic nutritional composition.

11: The method of claim 1, wherein the combination is administered in an infant formula, a starter infant formula, a follow-on or follow-up infant formula, a baby food, a growing-up milk, an infant cereal composition, a fortifier or a supplement.

12: The method of claim 1, wherein the combination is administered in a synthetic nutritional composition which comprises sphingomyelin in an amount ranging from 300 mg/kg to 820 mg/kg, phosphatidylcholine in an amount ranging from 500 mg/kg to 1.3 g/kg, phosphatidylinositol in an amount ranging from 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from 3.5 mg/100 g to 7 mg/100 g, Vitamin B12 in an amount ranging from 0.1 mcg/100 g to 10 mcg/100, folic acid in an amount ranging from 110 mcg/100 g to 400 mcg/100 g and DHA and ARA in an amount ranging from 30 mg/100 g to 300 mg/100 g.

13-19. (canceled)

20: The method according to claim 1 for promoting pleasure and enjoyment related to high stimulus intensity, rate, complexity, novelty and incongruity, and for reducing lowered mood and activity related to suffering physical state, object loss or inability to perform desired action.

21: The method according to claim 1, wherein the combination is administered during the first 36 months of life of the infant, toddler or young child.

22: The method according to claim 1, wherein mood and joy behaviours are promoted in the infant, toddler or young child between the age of 0 to 12 months.
